(19) United States
(12) Patent Application Publication
(10) Pub. No.: US 2021/0267474 A1
(43) Pub. Date: Sep. 2, 2021

(54) TRAINING METHOD, AND CLASSIFICATION METHOD AND SYSTEM FOR EEG PATTERN CLASSIFICATION MODEL

(71) Applicant: Wuyi University, Jiangmen (CN)

(72) Inventors: Hongtao Wang, Jiangmen (CN); Tao Xu, Jiangmen (CN); Guanyong Lu, Jiangmen (CN)

(21) Appl. No.: 17/004,832

(22) Filed: Aug. 27, 2020

(30) Foreign Application Priority Data
Mar. 2, 2020 (CN) 2020101361695

(51) Int. Cl. A61B 5/04; G06K 9/62; G06N 3/08; G16H 30/20; A61B 5/0476; A61B 5/00

(52) U.S. Cl. CPC A61B 5/04012; G06K 9/6277; G06K 9/6298; G06K 9/6257; G06K 9/00885; G16H 30/20; A61B 5/0476; A61B 5/7267; G06N 3/08

(57) ABSTRACT

A training method for an electroencephalogram (EEG) pattern classification model, including: acquiring EEG data, pre-processing the EEG data, and labeling the EEG data to obtain a labeled training data set, wherein the training data set comprises the pre-processed and labeled EEG data; inputting each piece of EEG data in the training data set into an attention-mechanism-based convolutional neural network to extract pattern features of the EEG data; and modifying parameters for the EEG pattern classification model according to the pattern features and labels of the EEG data.

### CROSS-REFERENCE TO RELATED APPLICATIONS

This application is based on and claims the benefit of priority from Chinese Patent Application No. 202010136169.5, filed on 2 Mar. 2020, the entirety of which is incorporated by reference herein.

### TECHNICAL FIELD

The present disclosure relates to the field of physiological digital information processing, and in particular, to a training method, a classification method and a system for an electroencephalogram (EEG) pattern classification model.

### BACKGROUND

With the advent of Internet economy, the sharing car is booming, which can be benefit to the one who has achieved a driving license. However, road traffic crash (RTC) is still a greater threat to our life than other human disease. The risk factors for RTC are various such as speed and driving behavior. And drowsiness and fatigue are likely to have a large contribution to RTC but difficulty in assessing their impact quantitatively. Objective and effective evaluation of the state of the driver seem to be more important for the organizer rather than simply verify their qualifications through the smartphone apps. Moreover, it is hard to make sure the real driver is the one who register at the app during the whole journey. And it is reported that the user used the forged certificate for the registration of being the user of sharing cars. Such kind of behavior brings great hazard to driving safety and thus the PI for such industry is becoming urgent. An easy way to personal identification (PI) during the journey is to monitor the driver with a camera without any consideration of privacy. Such a method also needs a high quality of the ambient light and an appropriate position of camera for most drivers.

With the development of deep learning, personal identification is upgraded from integrating various function into ID card to dynamic identification (DI). Many industries will benefit from such DI. For example, Factories can use DI to determine which workers are engaged in what process. In this way, enterprise can improve production efficiency and clarify the responsibility of an accident. On the other hand, biomedical signals are always used for disease diagnosis, mental state assessment and emotional related tasks. Thus, to do it in a more subtle way is good for the progress of the main task. Therefore, effective detection of fatigue state of the driver as well as simultaneous verification of the driver's identity along the journey are increasingly worth paying attention to.

### SUMMARY

The following is an overview of the subject matter described in detail hereinafter, which is not intended to limit the protection scope of the claims.

A training method, and a classification method and system for an EEG pattern classification model are provided in embodiments of the present disclosure, which can perform multitask classification on the same data on the premise of protecting privacy and can be applied to EEG-signal based biometric authentication and driving fatigue detection.

In an aspect, a training method for an EEG pattern classification model is provided in the embodiments of the present disclosure, comprising: acquiring EEG data, pre-processing the EEG data, and labeling the EEG data to obtain a labeled training data set, wherein the training data set comprises the pre-processed and labeled EEG data; inputting each piece of EEG data in the training data set into an attention-mechanism-based convolutional neural network to extract pattern features of the EEG data; and modifying parameters for the EEG pattern classification model according to the pattern features and labels of the EEG data.

In another aspect, a method for classifying EEG patterns is provided in the embodiments of the present disclosure, comprising: acquiring EEG signals, and pre-processing the EEG signals to obtain an EEG data set, wherein the EEG data set comprises the pre-processed EEG signals; inputting each EEG signal in the EEG data set into an attention-mechanism-based convolutional neural network to extract pattern features of the EEG data; and classifying the pattern features of the EEG data to obtain an EEG pattern classification result.

In yet another aspect, a system for classifying EEG patterns is provided in the embodiments of the present disclosure, comprising: a memory; a processor; a sensor connected to the processor, configured to detect the EEG signals; and a computer program stored in the memory and runnable on the processor, wherein when the processor executes the computer program, the method is implemented according to the EEG signals detected by the sensor.

A training method for an EEG pattern classification model, a method for classifying EEG patterns, and a system for classifying EEG patterns are provided respectively according to some embodiments of the present disclosure, for driving-related multitask classification which related to the PI as well as the driving state with the same data. The mean classification accuracy can be as high as 98.5% and 98.2% for PI and driving state, respectively. It can also make a good trade-off between the classification accuracy and the time cost. Our results manifest that the proposed network structure have the potential for multitask classification with biomedical signal for different applications.

Other features and advantages of the present disclosure will be set forth in the subsequent description and, in part, will become apparent from the description or may be understood by the implementation of the present disclosure. The objective and other advantages of the present disclosure may be achieved and obtained through the structure specified in the specification, claims and the accompanying drawings.

### BRIEF DESCRIPTION OF THE DRAWINGS

To better illustrate the technical solutions that are reflected in various embodiments according to this disclosure, the accompanying drawings intended for the description of the embodiments herein will now be briefly described, it is evident that the accompanying drawings listed in the following description show merely some embodiments according to this disclosure.

FIG. 1 is a flowchart of a training method for an EEG pattern classification model according to an embodiment of the present disclosure;

FIG. 2 is a schematic diagram of a CNN-Attention-based network according to an embodiment of the present disclosure;

FIG. 3A is an experimental scenario of a training method for an EEG pattern classification model according to an embodiment of the present disclosure;

FIG. 3B is a schematic diagram of sensors placed at specific locations on the scalp in a training method for an EEG pattern classification model according to an embodiment of the present disclosure;

FIG. 3C illustrates the averaged mean reaction time of awake and fatigue state for all 31 subjects in a training method for an EEG pattern classification model according to an embodiment of the present disclosure;

FIG. 4A illustrates PI classification accuracy for all 31 subjects with the error bar manifests that a 10-fold cross validation method applied to such classification;

FIG. 4B illustrates comparison of PI classification accuracy with four methods for PI classification accuracy;

FIG. 4C illustrates comparison of PI classification accuracy of one subject with four methods, wherein the lowest mean accuracy which belongs to Subject 1 in FIG. 4A is chosen;

FIG. 4D illustrates comparison of time cost of PI classification with four methods;

FIG. 4E illustrates comparison of loss function with four methods of PI classification;

FIG. 5A illustrates the fatigue state accuracy for all 31 subjects with a training method for an EEG pattern classification model according to an embodiment of the present disclosure, in which the error bar manifests that a 10-fold cross validation method applied to such classification;

FIG. 5B illustrates comparison of fatigue state accuracy with four methods. Each bar stands for the averaged accuracy of 10-fold cross validation results of all 31 subjects;

FIG. 5C illustrates comparison of time cost of fatigue state classification with four methods;

FIG. 5D illustrates comparison of loss functions with four methods for classifying fatigue and awake states;

FIG. 5E illustrates the Fatigue state accuracy of subject 12, wherein subject 12 achieved the lowest mean fatigue state accuracy with Attention network;

FIG. 5F illustrates the fatigue state accuracy of subject 31, wherein subject 31 achieved the lowest mean fatigue state accuracy with CNN network;

FIGS. 6A to 6D illustrate different configurations of a small number of electrodes for the classification of PI and driving fatigue state, wherein:

FIG. 6A illustrates a small number of electrodes in different configurations according to an embodiment of the present disclosure, placed in the occipital and parietal lobes (OP);

FIG. 6B illustrates a small number of electrodes in different configurations according to an embodiment of the present disclosure, placed on the front (F);

FIG. 6C illustrates a small number of electrodes in different configurations according to an embodiment of the present disclosure, placed in the center and parietal lobe (CP);

FIG. 6D illustrates a small number of electrodes in different configurations according to an embodiment of the present disclosure, placed in the frontal and parietal lobes (FP);

FIGS. 7A to 7D illustrate comparison of results of classification accuracy with a small number of electrodes according to an embodiment of the present disclosure, wherein

FIG. 7A illustrates averaged mean PI classification accuracy with different channels (equivalent to signal channels of sensors at different positions);

FIG. 7B illustrates the average PI classification accuracy of different channels of Subject 28;

FIG. 7C illustrates averaged mean driving fatigue state classification accuracy with different channels;

FIG. 7D illustrates the highest mean driving fatigue state classification accuracy for different subjects with different channels;

FIGS. 8A to 8D illustrate the Pearson correlation between the mean accuracy of PI and the mean accuracy of driving fatigue state, according to an embodiment of the present disclosure, wherein

FIGS. 8A to 8D illustrate ATT-CNN; LSTM-CNN; CNN; ATT respectively;

FIG. 9A to 9D illustrate comparison of PI classification with fatigue data alone and with mixed data according to an embodiment of the present disclosure, wherein

FIG. 9A illustrates comparison of the PI classification accuracy;

FIGS. 9B-9C illustrate the Pearson correlation between the mean accuracy of PI and the mean accuracy of driving fatigue state with fatigue and mixed data;

FIG. 9D illustrates the time cost comparison with different data (awake, fatigue and mixed);

FIGS. 10A-10B illustrate comparison of the PI classification accuracy under different network kernel sizes for a neural network adopted according to an embodiment of the present disclosure; and

FIG. 11 illustrates a flowchart of a method for classifying EEG patterns according to an embodiment of the present disclosure.

### DETAILED DESCRIPTION

Embodiments of the present disclosure will be described in detail in the following descriptions, examples of which are shown in the accompanying drawings, in which the same or similar elements and elements having same or similar functions are denoted by like reference numerals throughout the descriptions. The embodiments described herein with reference to the accompanying drawings are exemplary, which are used to explain the present disclosure, and shall not be construed to limit the present disclosure.

Reference throughout this specification to “an embodiment”, “some embodiments”, “one embodiment”, “an example”, “a specific example,” or “some examples” means that a particular feature, structure, material, or characteristic described in connection with the embodiment or example are included in at least one embodiment or example of the present disclosure. In the specification, expressions of the above terms are not necessarily referring to the same embodiments or examples. Furthermore, the feature, structure, material, or characteristic described can be incorporated in a proper way in any one or more embodiments or examples. In addition, under non-conflicting condition.

It should be understood that in the description of embodiments of the present disclosure, “multiple” means more than two, “greater than”, “less than”, “more than”, etc., are understood as not including the number itself, while “above”, “below”, “within”, etc., are understood as including the number itself. The terms of “first”, “second” and the like are only used to distinguish technical features, and should not be understood as indicating or implying relative importance or implicitly indicating the number of technical features indicated or implicitly indicating the precedence of the technical features indicated.

The driving fatigue detection method takes advantage of extracting different features such as physiological features (EEG, electrocardiogram (ECG) and electromyography (EMG) and electrooculogram), driver's performance (facial express) and vehicle's state, and the combination of the aforesaid features. For example, the detection of vehicle's state depends on the analysis of the sensor signals processed by electrical control unit (ECU) of a vehicle. In this stage, steering wheel motion and lane departure detection are main methods for driving fatigue detection. However, such methods are affected by road information, which are only useful in a certain environment. In addition to indirectly detection of vehicle state for driving state, a more direct method, facial expression detection, is always used in distinguishing the fatigue state of a driver. For example, the visual cues like eyeblink, head movement and yawn emotion were recorded and used for developing classification model of driving fatigue detection. However, the biggest limitation of these methods lies in that they are greatly affected by environmental light. In addition, fusing more features can improve the reliability of fatigue state recognition while increasing the complexity of data acquisition and classification system. However, physiological features always provide more objective information for driving fatigue detection as an individual can exert little control on them. Therefore, electrophysiological signals like EOG, EEG, ECG and EMG which can exclude the road and light impact and indicate the mentality of subjects in real-time has attracted many interests. Among numerous electrophysiological indicators available for estimating driving fatigue state, EEG signals have been proven to be a robust one. And compositions (alpha, delta and theta wave) within such signal are highly corrected with fatigue states.

As mentioned herein, PI with a private way is also significant for such sharing economy as it can benefit the business promotion like big-data-precise-push. More importantly, sharing economy with PI function can be convenient for the public, and be conductive to accountability minimizing the loss of the company. On the other hand, requests for the identification of living persons is becoming common and the most commonly used means of PI is surveillance system with image or video recording. However, such a system always serves for public safety and is controlled by national security agency exclusively. In hence, it is hard for business organization to access the related network although it is quite necessary to do so. Apart from surveillance system, biometrics, which uses distinctive features of human body for PI, is attracting many interests. The traditional biometrics includes fingerprint, iris, face and even gait. However, such biometrics is not suitable for sharing car. For example, biometrics like fingerprint can be forged. In addition, the most important issue lies in that the identification process is better to be a long term one which can be throughout the whole journey. Therefore, physiological signals which have both merits of long-term recording and protecting the privacy attract attentions. In view of the robust property of EEG for fatigue state classification, it is suspected that if the unique biometric characteristics of EEG signal could be used to realize PI. And such a study can satisfy both requirement of identifying driving fatigue state and the person for sharing cars. Therefore, according to some embodiments of the present disclosure, training method, classification method and system are provided, for both driving fatigue detection and PI.

Due to the unique feature of some kinds of biomedical signal of an individual, such a signal for both PI as well as biomedical related tasks may be used. In this paper, electroencephalography (EEG) signal is used for both the PI and the fatigue state detection during driving. Such an EEG based method adopted an attention-based convolutional neural network (CNN) which has a high spatiotemporal resolution. The accuracy of PI can reach 98.5% while the accuracy of fatigue state during driving can be as high as 97.8%. The significance of our results lies in, using a deep learning method for the multitask classification with the same data, according to some embodiments of the present disclosure. In the future, the proposed method may have the potential to let biomedical signal be developed as an encryption for the protection of privacy.

CNN is a useful tool which has been widely used in the pattern recognition such as image recognition, classification of handwritten, natural language processing and face recognition. The connectivity between neurons in the CNN was like the organization of the animal visual cortex which makes CNN remarkable in pattern recognition. CNNs are a specialized kind of neural network for processing input data that has an inherent grid-like topology. In another word, the nearby entries for the input data to CNN are correlated and the example of this kind of input is the 2-dimension image. Therefore, CNN has been increasingly applied in pattern-related biomedical applications. For example, the animal behavior classification, the skin cancer diagnosis, protein structure prediction, electromyography (EMG) signal classification and ECG classification. In this study, EEG signals which are recorded with 24 sensors on the subject's scalp should have inherent correlation between sensors. Hence, CNN is used to distinguish the driving fatigue state with recorded EEG signals. On the other hand, CNN is superior in automatically doing feature extraction involving large datasets.

EEG is a kind of temporal sequence between which two consecutive moments are correlated. However, traditional CNN do not have memory mechanism that can process the correlation of sequential inputs, leading to the loss of information. Hence, in this study, the attention mechanism is combined together with CNN. Such a mechanism is always used in natural language processing for the modelling of long-term memory. The underlying logic of our model believe that not all channel signals contribute equally to related classification, and the correlation within one channel signal involves in the PI or fatigue state detection.

According to the embodiments described hereinafter of the present disclosure, several aspects are introduced, which includes the experiment and data acquisition, signal preprocessing, and the classification for both PI and driving fatigue states, results of classification, comparisons with other methods, etc.

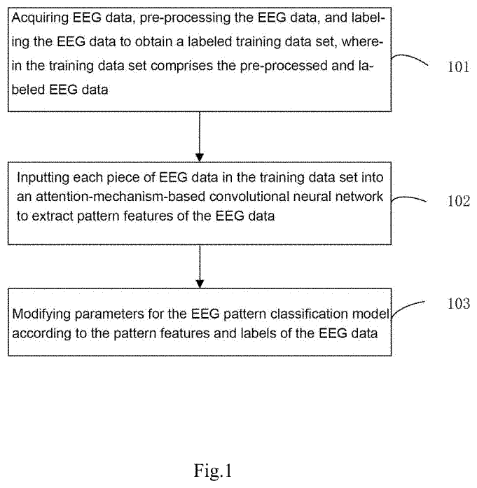

As shown in FIG. 1, a flowchart of a training method for an EEG pattern classification model according to an embodiment of the present disclosure includes without being limited to the following steps:

step 101: acquiring EEG data, pre-processing the EEG data, and labeling the EEG data to obtain a labeled training data set, wherein the training data set includes the pre-processed and labeled EEG data;

step 102: inputting each piece of EEG data in the training data set into an attention-mechanism-based convolutional neural network to extract pattern features of the EEG data; and

step 103: modifying parameters for the EEG pattern classification model according to the pattern features and labels of the EEG data.

The model can be used for both PI and fatigue state detection tasks during driving.

In some embodiments, in step 101, the EEG data is obtained by sensors. In some other embodiments, sample EEG data for training a model can be obtained directly from an existing medical database.

In an exemplary embodiment, obtaining EEG data for training a model by sensors may specifically include:

acquiring EEG signals from multiple EEG signal sensors;

obtaining a multi-channel EEG signal by performing bandpass filtering and Fast ICA on the EEG signals;

digitizing and segmenting the multi-channel EEG signal according to a preset sampling rate and duration to obtain an EEG data set including multiple multi-channel EEG signal digital segments;

adding at least one label to each multi-channel EEG signal digital segment in the EEG data set to obtain labeled EEG data, wherein the label includes an awake state, a fatigue state, and a driver ID; and

obtaining the labeled training data set.

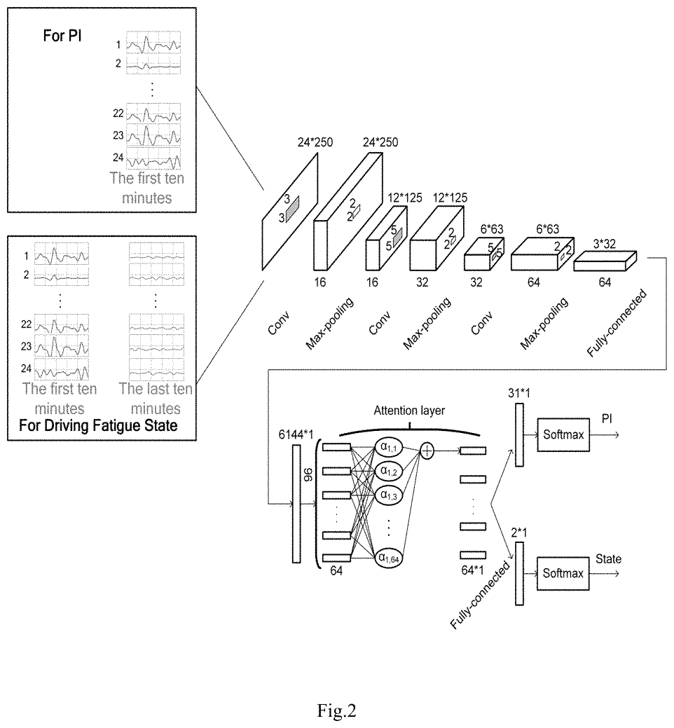

When the attention-mechanism-based convolutional neural network (hereinafter referred to as Att-CNN, or as CNN-Attention-based network, as shown in FIG. 2) is used for PI and driving fatigue state classification, standard sample data can be obtained by having multiple subjects as drivers through normalized experimental simulation scenarios as described below. In general, for example, an experiment of each subject lasts 50 minutes. By comparing the average response time of all subjects, the first 10 minutes can be defined as an awake state and the last 10 minutes as a fatigue state. For PI, the EEG data in the awake state can be input into the structure. Alternatively, the EEG data in a mixed state (awake and fatigue) can also be input into the network for PI classification. Besides, the EEG data in two states (awake and fatigue) is input into the network to classify the driving fatigue state and the awake state.

The collected EEG data may have a multiplexed signal (for example from 24 sensors placed on the scalp of a subject) with a sampling rate of 250 Hz. The input of the network may be a 1 second duration collected signal (one label) with a size of 24*250 without any overlap.

According to requirements of training and testing, 90% EEG signals are chosen from the sample set as the training dataset and the left 10% are used as test pattern (or referred to as test set) for performance evaluation. For the detection of driving fatigue state, the experimental time for each subject is 20 mins (for example, the first 10 minutes plus the last 10 minutes during a total 50 minutes) and thus each subject has 1200 (20×60) labels. And for PI, only feed 10 mins signal to the network and thus each person has 600 labels. The total training epochs was set to be 500 and 30 for the classification of PI and driving fatigue state, respectively.

Then, the marked training data set is fed into the Att-CNN as shown in FIG. 2 and table 1 for PI and driving fatigue state classification. As shown in FIG. 2, different data is input into the Att-CNN structure for the PI and driving fatigue state classification.

TABLE 1
Structure of the neural network

| Type | Filter | Size/Stride | Input | Output |
|---|---|---|---|---|
| Conv 1 | 32 | 3*3/1 | 24*250 | 24*250*16 |
| Max-pool 1 | | 2*2/2 | 24*250*16 | 12*125*16 |
| Conv 2 | 64 | 5*5/1 | 12*125*16 | 12*125*32 |
| Max-pool 2 | | 2*2/2 | 12*125*32 | 6*63*32 |
| Conv 3 | 128 | 5*5/1 | 6*63*32 | 6*63*64 |
| Max-pool 3 | | 2*2/2 | 6*63*64 | 3*32*64 |
| ATT | | | 64*96 | 64*1 |
| Fully cnnected | | | 64*1 | 31*1 or 2*1 |
| Softnnax | | | 31*1 or 2*1 | Probability |

where Cony represents a convolution layer, Max-pool represents a max-pooling layer, and Fully connected represents a fully connected layer.

In some embodiments, the Att-CNN adopted in the present disclosure includes: at least one convolution layer; at least one max-pooling layer; an attention module; and a fully connected layer;

wherein the inputting each piece of EEG data in the training data set into an attention-mechanism-based convolutional neural network to extract pattern features of the EEG data includes:

inputting each piece of EEG data into the at least one convolution layer, and extracting the pattern features of the EEG data to obtain a convolution feature vector including the pattern features;

inputting the convolution feature vector into the at least one max-pooling layer for pooling to obtain a pooled feature vector;

inputting the pooled feature vector into the attention module to calculate a normalized weight for the pooled feature vector and a sum of information reflecting the pattern features of the EEG data; and

outputting the pattern features of the EEG data through the fully connected layer.

In this network structure, there may be three convolution layers, in which convolution kernels may have different sizes. Each convolution layer can be regarded as a fuzzy filter, which enhances original signal characteristics and reduces noise, and can be expressed as:

xⱼ [symbol] =f(Σ_{tϵM}_{ⱼ}W [symbol] j [symbol] ×x [symbol] −1+bⱼ [symbol] ), (1)

where xⱼ [symbol] stands for a feature vector corresponding to the first convolution kernel of the jth convolution layer with a size of 16*24*250; and f(·) stands for an activation function. According to the embodiment of the present disclosure, Swish may be used as the activation function because it has better nonlinearity than a rectifying linear unit (ReLU).

f(x)=x·Sigmoid(βx), (2)

where β is a constant that equals to 1; Mj represents accepted domain of a current neuron, and denotes the ith weighting coefficient of the jth convolution kernel in the first layer; bjl represents an offset coefficient corresponding to the jth product of the first layer.

The performance comparison of network structures with different convolution kernel sizes will be further discussed later in the following parts. In the convolution layer, a feature vector of an upper layer is convoluted with a convolutional kernel of a current layer. The result of the convolution operation passes through the activation function and then forms a feature map of this layer. Each convolutional layer corresponds to a pooling layer (maximal pooling) which retains useful information while reducing data dimensions.

In some embodiments, the CNN-Attention-based Network takes advantage of encode-decode frame in which CNN acts as an encoder and attention mechanism is the decoder. In the present disclosure, it is believed that EEG is a kind of temporal sequence in which signals are temporally correlated. And attention focuses on the extraction of important segmentation of EEG signals which can represent the feature of the person or the state. The structure of attention is shown in Error! Reference source not found. and table 1. After the fully connected layer of CNN, the EEG signal is rearranged into a 96*64 matrix (hi) which is similar to the sentence encoder of sentence attention. Each line of hi corresponds to i sentences. The attention mechanism can be expressed as

$$\begin{matrix} {u}_{i} = tanh ( W \text{?} h \text{?} + b \text{?} ) & ( 3 ) \\ {a}_{i} = \frac{exp ( {u}_{i}^{T} u \text{?} )}{{\sum }_{i} exp ( {u}_{i}^{T} u \text{?} )} & ( 4 ) \\ v = {\sum }_{i} {a}_{i} {h}_{i} \\ \text{?} \text{indicates text missing or illegible when filed} & ( 5 )\end{matrix}$$

where bs is the bias. ui is a hidden representation of hi which is fed through a one-layer perceptron with the weight Ws. αi is a normalized importance weight which is measured by the similarity of ui with us. us is a hidden representation of another piece of EEG signal (one line of hi). After that, one can get v which is the summation of the all information of EEG signals.

Softmax can solve multiple classification problem and thus one can use such a classifier for both PI and driving fatigue state classification. According to different testing input x, the probability value p manifests the classification result. The hypothesis function yields a 31-dimensional vector or a 2-dimensional vector for PI or driving fatigue state, respectively.

In some embodiments, the Att-CNN of the present disclosure may further include: a Softmax classifier placed after the fully connected layer, configured to classify the driver ID PI, and/or classify the awake-state and fatigue-state pattern features of the driver, wherein feature vectors of the pattern features of the EEG data are input to the classifier, and EEG pattern classification results are output by calculation based on a function h_{θ}(x) of the classifier, wherein the function h_{θ}(x) of the Softmax classifier is expressed as:

$${h }_{θ} ( x \text{?} ) = [ \begin{matrix}p ( y \text{?} = 1 \vert x \text{?} ; θ ) \\ ⋮ \\ p ( y \text{?} = k \vert x \text{?} ; θ )\end{matrix} ] = \frac{1}{{\sum }_{j = 1}^{k} e \text{?}} [ \begin{matrix}e \text{?} \\ ⋮ \\ {e}^{\text{?}}\end{matrix} ] \\ \text{?} \text{indicates text missing or illegible when filed} \qquad ( 6 )$$

where x is a function input, θ₁, θ₂ . . . θₖ∈ [symbol] ⁿ⁺¹ denotes model parameters, for example, parameters for extracting features, and k is a classification dimension, for example, k=31 or 2, which, according to a PI and awake fatigue state classification task, can represent 31 drivers to be identified or the awake and fatigue states, respectively.

$$\frac{1}{{\sum }_{j = 1}^{k} e \text{?}}$$

$$\text{?} \text{indicates text missing or illegible when filed}$$

normalized the probability distribution so that the summation of probabilities is 1., i.e., the sum of respective vector elements is 1, wherein the value with higher probability is taken as a classification result.

To accelerate the training, cross entropy can be used as a cost function of this CNN, which can be expressed as a loss function L:

$$L = - \sum \text{?} y \text{?} log ( {h}_{θ} ( x \text{?} ) ) , \\ \text{?} \text{indicates text missing or illegible when filed} \qquad ( 7 )$$

where y is an output vector, and h_{θ} is a probability of belonging to a category of classification.

A learning algorithm of the above network structure is as shown in table 2.

TABLE 2
The algorithm for the training of cnn-attention-network
Algorithm 1: Training of CNN-Attention-Network

| | |
|---|---|
| Labeled training dataset | (A^{(s)} is the s^{th} training dataset and yₛ is |
| {(A^{(s)}, y [symbol] ; | the label corresponding to A^{(s)}. |
| CNN-Attention-based | θ is the model parameters and A is the |
| Network model [symbol] (A; θ); | all the training dataset. |
| Loss function L(y, ŷ); | y is labels of all training dataset and ŷ |
| | is the estimated y. |

```
Number of optimization epochs J;
N-batch size 256;
Output: Learned parameters θ for the model [symbol]  (A; θ).
Initialize parameters θ;
  for j = 1 : J do
    Extract number of N-batches (256) of samples from A^{(s)}
    Ã^{(s)} ← Permute the rows of A^{(s)}
    for i = 1: n (n = 31 or 2) do
      Permute the entries of Ã^{(s)}ₜ;
    end
    Update θ^{(j)} via Adam optimizer for the loss function in (4);
end [symbol]
indicates data missing or illegible when filed
```

A scenario for Att-CNN model training according to the present disclosure will be described below. According to the present disclosure, The original intention of this work aims to the study of driving fatigue state. Therefore, to effectively represent the driving fatigue state of subjects, the driving fatigue experiment is carefully designed so that one can acquire the valuable data efficiently. To achieve a more authentic driving experience, arrange the environment (light, sound effect, etc.) as real as possible so that the subject could feel they are indeed in an expressway. In addition, to reduce the complexity of the assessment, only consider the time factor for each subject rather than other elements like subjects' cooperative attitude. In this section, the subjects, simulated driving environment, awake and fatigue state judgement and data acquisition will be introduced.

1) Subjects

31 subjects whose average age are 23 are employed in this study. Each subject should have considerable driving experience and be familiar with the simulated driving environment. Furthermore, each subject was forbidden to absorb coffee and alcohol within 4 and 24 hours respectively before the experiment. The subject should have a good sleep the night before the experiment. In addition, they should clean up the hair to avoid inducing excessive resistance for the sensor during the EEG signal acquisition. Before conducting the experiment, they are given a period of time to be familiar with the system eliminating operational errors.

2) Simulated driving environment

According to some embodiments of the present disclosure, the experiment may be conducted in a virtual reality environment as it is dangerous to drive on an expressway accompanying with a distracting experiment. The virtual reality simulated driving environment is consisted by a simulated driving system and a wireless dry EEG acquisition system (Cognionics headset HD-72). The simulated driving system are equipped with three 65-inch LCD screens, a Logitech G27 Racing Wheel simulator (a driving wheel, three pedals, and a six-speed gearbox) and a host computer which provides a driving environment (Error! Reference source not found.3A). To provide a more realistic sense of driving, the experiment is conducted in dark surroundings and the incident light is from the three 65-inch LCD screens which monitors two-sided rearview mirror, dashboard, and an expressway with a sunny day.

3) Awake and fatigue state judgment

The experiment which lasts for 40 or 50 mins is arranged, for example, between 3 pm to 5 pm when the subject is prone to suffer from fatigue. During the experiment, the driver will randomly receive brake signal elicited from the guide vehicle with the lighting up of the rear lamp. To be more objective access to driver's fatigue state, one may use the reaction time to indicate the subject's driving fatigue state. The reaction time which will decrease with the experiment goes on is defined as the onset of the lighting up of the rear lamp to the stepping of the brake pedal. Experimental evidence manifests that the transition from the alert to fatigue state during driving lasts for about 30 mins and there is a significant difference of the averaged mean reaction time between the first ten mins and the last ten mins of the experiment (Error! Reference source not found.3C). Hence, one may define the EEG data of the first ten minutes and the EEG data of the last ten minutes as the awake state and the fatigue state, respectively.

4) Data Acquisition

EEG signals are collected by Cognionics headset which distributed 24 sensors on the subject's scalp (Error! Reference source not found.3B). The impedance of sensors is below 20 kΩ. The collected EEG signal was sampled at 250 Hz and filtered with a bandpass filter (0.5-100 Hz). After that, such collected signals are transmitted to a laptop (Toshiba Intel(R) Core (TM) i5-6200U Duo 2.4 GHz) by a Bluetooth module for further data analysis

Experimental and model training results

1) PI Classification

During model training, EEG signals are collected from 31 subjects each of which conducted the experiment for 40, 50 or 90 mins. And only take data of the first 10 mins and the last 10 mins from a complete experiment for further analysis. For each subject, one may randomly chose 90% and 10% of the total labelled data as the training set and testing set, respectively. First, do the classification of PI for each subject with the CNN-Attention-based network and a 10-fold cross validation method is used for such a classification (Error! Reference source not found.4A). The accuracy of 4 subjects (Subject 17, 18, 21, 22) reached 100%. The lowest mean accuracy can be as high as 96.3% (Subject 1). Then evaluate the performance of the CNN-Attention-based network by comparing the classification accuracy with other three methods for each subject. One may use the same preprocessing method and the classifier for this comparison. As is shown in Error! Reference source not found.4B, the mean classification accuracy of PI for all 31 subjects were averaged. The mean accuracy of CNN-Attention-based network can reach 98.5% which is higher than three other methods (CNN-LSTM: 95.3%; CNN: 91.9%, Attention: 71.2%).

As the mean classification accuracy of Subject 1 with CNN-Attention-based network is the lowest among that in all 31 subjects, one may compare the performance of Subject 1 with four methods (Error! Reference source not found.4C). CNN-Attention-based network achieve a highest mean classification performance (96.3%) with a minimal STD (0.0246). Such a result manifest that the classification of PI with CNN-Attention-based network have a relative higher and more stable performance than that of other network structures. Apart from showing the classification accuracy of CNN-Attention-based network, the running time of such a model is also compared with other methods (Error! Reference source not found.4D). It only takes 1.86s for each epoch with the proposed neural network while it takes more than twice as much time to run one epoch with LSTM-based CNN (4.4s) (or referred to as CNN-LSTM herein). Therefore, it is believed that one can get a good trade-off between the classification accuracy and the running time with the proposed method. In addition, the comparison of loss function of the four methods and CNN-Attention-based network can gradually converge to 0 after 150 iterations are shown.

2) Driving Fatigue State Classification

The classification of driving fatigue state is implemented for each subject with the CNN-Attention-based network and a 10-fold cross validation method is used for such a classification (Error! Reference source not found.5Error! Reference source not found.A). The lowest mean accuracy can be as high as 94% (Subject 12). Error! Reference Source not Found.5B Show the Comparison of averaged fatigue state accuracy with four methods and the proposed method can reach 97.8%. Then it is found that the person who get the lowest mean accuracy of fatigue state with CNN-Attention-based and CNN-LSTM-based network, respectively (Error! Reference source not found.E and Error! Reference source not found.F). Subject 2 got the worst mean accuracy with CNN-Attention-based network (94%) while Subject 31 achieved a much lower mean accuracy with CNN-LSTM-based network. Although the accuracy of subject 31 is the lowest among that of all subjects, it is much higher with a smallest STD than other methods, reflecting a small influence of input data to such a network structure. The time cost of four methods are compared (Error! Reference source not found.C). It only takes 0.18s to complete one epoch computation with CNN-Attention-based network which is even faster than that merely with CNN. Error! Reference source not found.D shows the comparison of loss function of the four methods for driving fatigue state classification. The convergence of the proposed method can be fast and stable comparing with other three methods.

Results of a Small Number of Electrodes

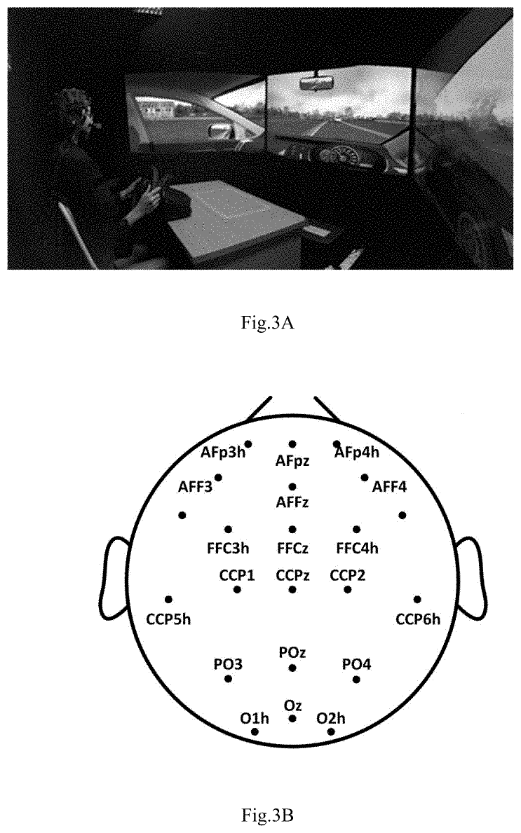

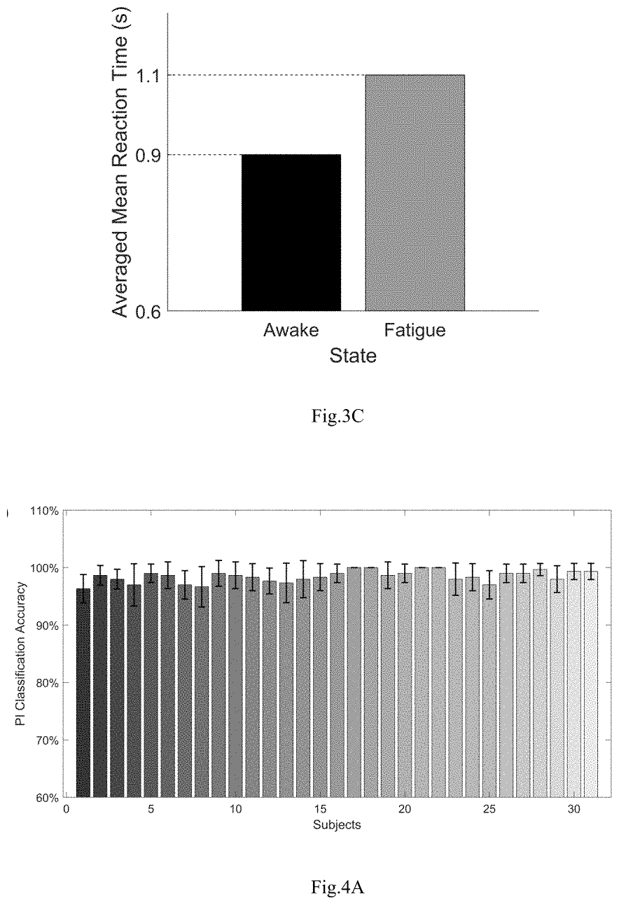

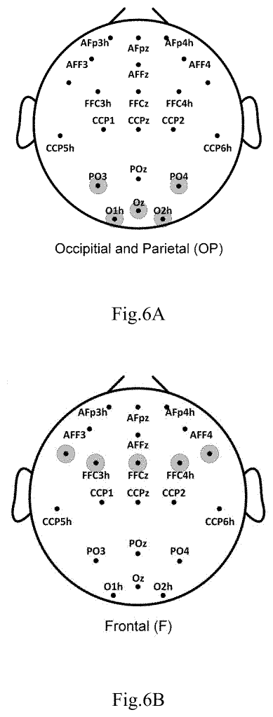

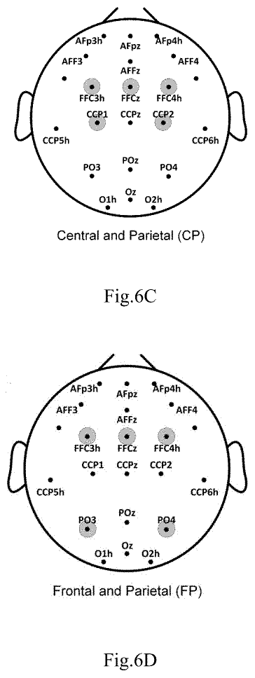

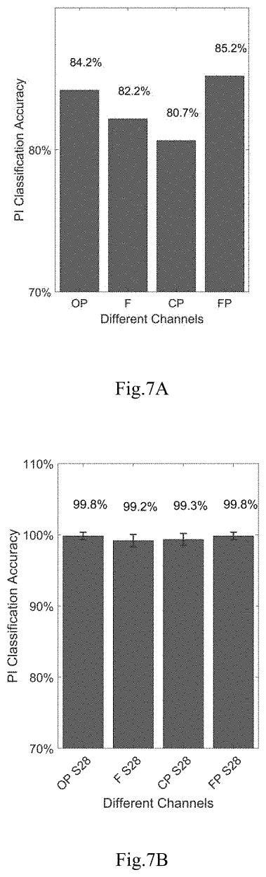

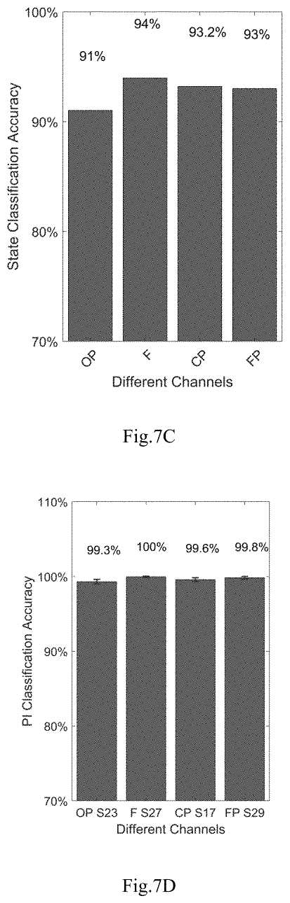

In some embodiments, the proposed network structure was also tested using a smaller number of electrodes than that in FIG. 3 for training a PI and driving fatigue state classification model. It is believed that applications with a small number of electrodes and acceptable classification accuracy could bring great convenience to users. The structure of a few electrodes is as shown in FIGS. 6A to 6D and the simulation results are as shown in FIGS. 7A to 7D. In a cathode protection zone, the average PI classification accuracy of five electrodes reached at least 80.7%. The classification accuracy of PI (Subject 28) was up to 99.2%. Besides, for all configurations of selected electrodes, the average classification accuracy of the driving fatigue state could be higher than 91%, and the highest could be 100% (the front of Subject 27).

Correlation Between Driving Fatigue State and PI

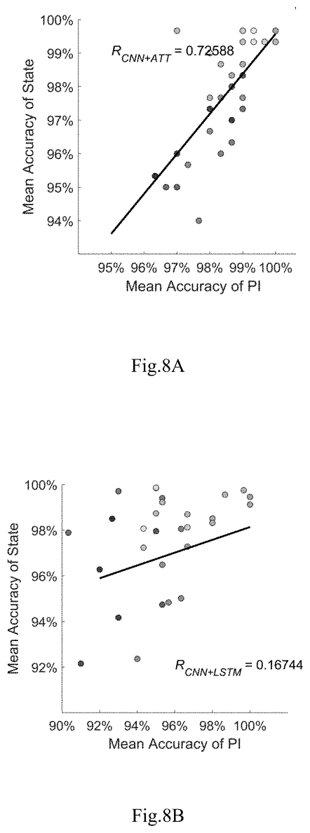

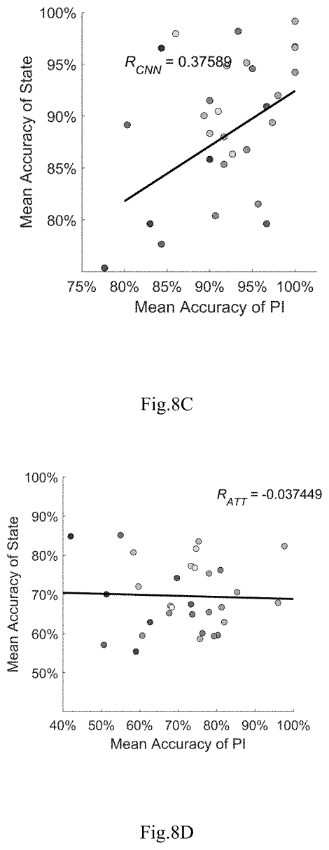

Finally, Pearson correlation was performed between the average accuracy of PI and the average accuracy of the driving fatigue state, as shown in FIGS. 8A to 8D. The Pearson correlation can be more than 0.72 manifesting a high correlation between the classification accuracy of PI and state.

Discussion of Training Results

According to an embodiment of the present disclosure, a

CNN-Attention-based network is developed for both driving fatigue state classification and PI with EEG signals. Specifically, 24-channel EEG signals from a subject who participate in a simulated driving environment are collected. After bandpass filtering with 0.5-100 Hz and preprocessing with FastICA, the data were transmitted to CNN-Attention-based network for dual tasks. Aspects of multitask learning, network kernel size, and other EEG-based applications will be discussed.

1) Multitask Learning

Traditional machine learning based multitask learning aims to make full use of information in related tasks to improve the overall performance of all tasks. For example, the speech recognition is to extract useful information in different circumstances regardless of an individual's pronunciation. Apart from speech recognition, multitask learning has many other applications such as computer vison, bioinformatics and health informatics, web applications and so on. The multitask learning is always achieved by sharing feature or model parameters among different tasks. And such tasks are related. However, in some embodiments of the present disclosure, these two classification tasks are derived from the same event (for example, a driver is driving) and thus the input data is shared and the same network structure is used for dual-task classification. The proposed multitask learning have more practical significance.

In some embodiments, a first EEG recognition model is trained according to the marked training data set including at least the driver ID label, wherein the first EEG recognition model is configured to identify and identify a driver PI based on EEG pattern features of a driver; and/or

a second EEG recognition model is trained according to the marked training data set including at least the awake state and fatigue state labels, wherein the second EEG recognition model is configured to classify awake-state and fatigue-state pattern features of the driver based on the EEG pattern features of the driver.

The result of PI classification with the EEG signal during the awake state is shown in Error! Reference source not found.4. To show the classification ability of the proposed network structure, the EEG signals during the fatigue state and the signal with both states (mixed state) are used to do the PI classification. Error! Reference source not found.9A shows the comparison of PI classification accuracy between the EEG signal of fatigue state and mixed state. The averaged mean accuracy of all 31 subjects with fatigue state input can reach 98% which is 10% higher than that with mixed signal. The Pearson correlation between the mean accuracy of state and the mean accuracy of PI with the two types of input data is shown. Rfatigue and Rmixed can reach 0.776 and 0.475. Such a result manifest that the PI and state classification have a high correlation with the proposed network structure. The time cost with three different kinds of input are compared. The time cost with awake EEG signal is almost the same as the one with fatigue EEG signal while the time cost with mix data is less than double of that with awake or fatigue data. It is because all the awake EEG signals as well as the fatigue EEG signals are fed into the network structure.

From simulation results (Error! Reference source not found.4 and Error! Reference source not found.5), the performance of CNN with attention mechanism or CNN with LSTM mechanism is much better than that with merely CNN or attention mechanism. Large numbers of literatures believe that CNN is superior in learning spatial hierarchies of features whereas the attention mechanism or LSTM is good at processing temporal sequence. Hence, by combining the two modalities can allow the network to achieve higher accuracy of classification. Attention-based CNN is preferred rather than LSTM-based CNN due to the attention mechanism allows the decoder to selectively pay attention to the information. However, if the source sequence is too long with large information, it will take more time for the encoder to condense the information into fixed length of representation. As is shown in Error! Reference source not found.4D and Error! Reference source not found.5C, it takes more than twice as much time for LSTM based CNN. In addition, it is noticed that in Error! Reference source not found.5C, it takes less time for the proposed neural network to complete one epoch computation than that with merely CNN. It is because in the fully connected layer it transforms from a 64×32×3 matrix to a 2×1 one for the CNN while it only transforms from a 64×1 matrix to a 2×1one for the proposed method.

Network kernel size

In an embodiment of the ATT-CNN network structure proposed in the present disclosure, only three convolutional layers are used to make a trade-off between the training time and the classification accuracy. Therefore, classification accuracy of PI with different size of convolutional kernel are compared (Error! Reference source not found.10). The averaged mean accuracy with the proposed kernel size is the highest one (Error! Reference source not found.10B). And it is shown that the lowest mean classification accuracy with its STD of different kernel size for different convolutional layers (Error! Reference source not found.10A). Subject1 achieves the high accuracy (96.3%) with a minimal STD (0.0246).

EEG-Based Application

After an EEG pattern classification model is trained using the training method for the EEG pattern classification model according to some embodiments of the present disclosure and optionally verified using a test set, it can be used as the PI described in the present disclosure, as well as the awake and fatigue state classification task.

According to an embodiment of the present disclosure, a method for classifying EEG patterns is proposed, which can be used mainly for PI and fatigue state detection tasks during driving.

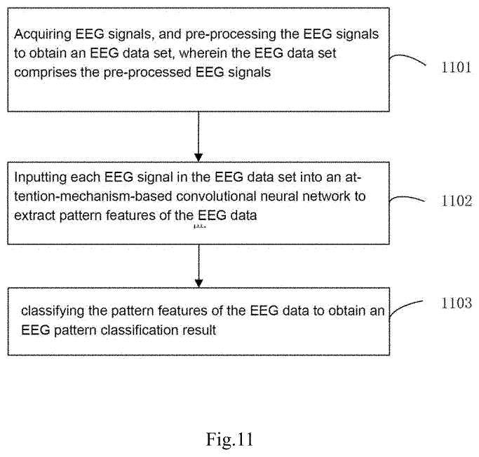

As shown in FIG. 11, the method includes, but is not limited to,

step 1101: acquiring EEG signals, and pre-processing the EEG signals to obtain an EEG data set, wherein the EEG data set includes the pre-processed EEG signals;

step 1102: inputting each EEG signal in the EEG data set into an attention-mechanism-based convolutional neural network to extract pattern features of the EEG data; and

step 1103: classifying the pattern features of the EEG data to obtain an

EEG pattern classification result.

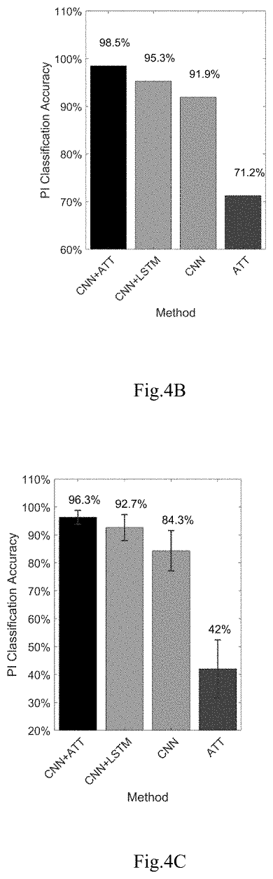

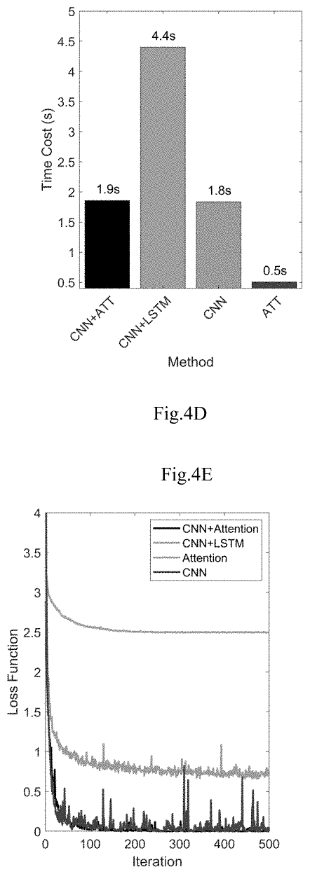

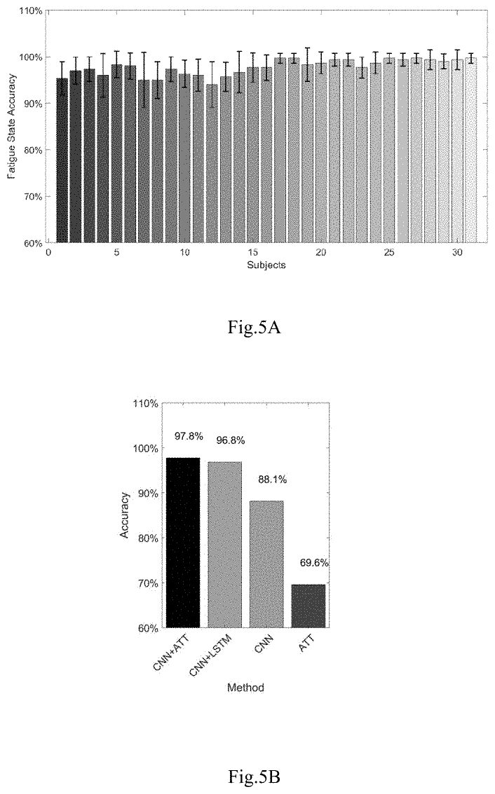

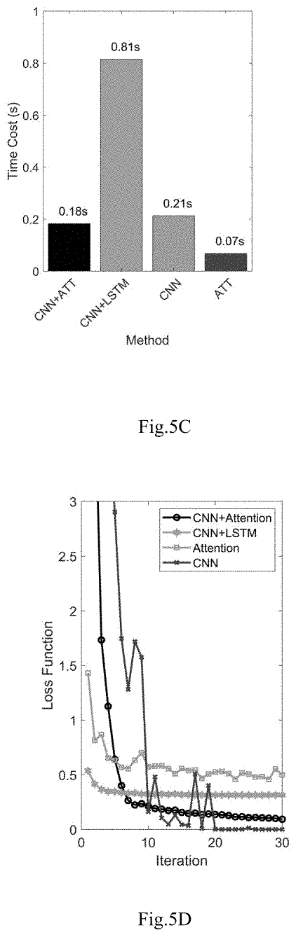

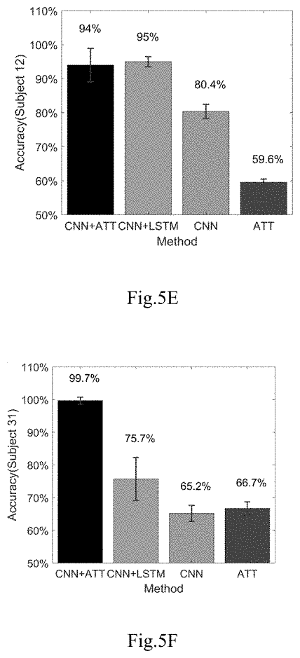

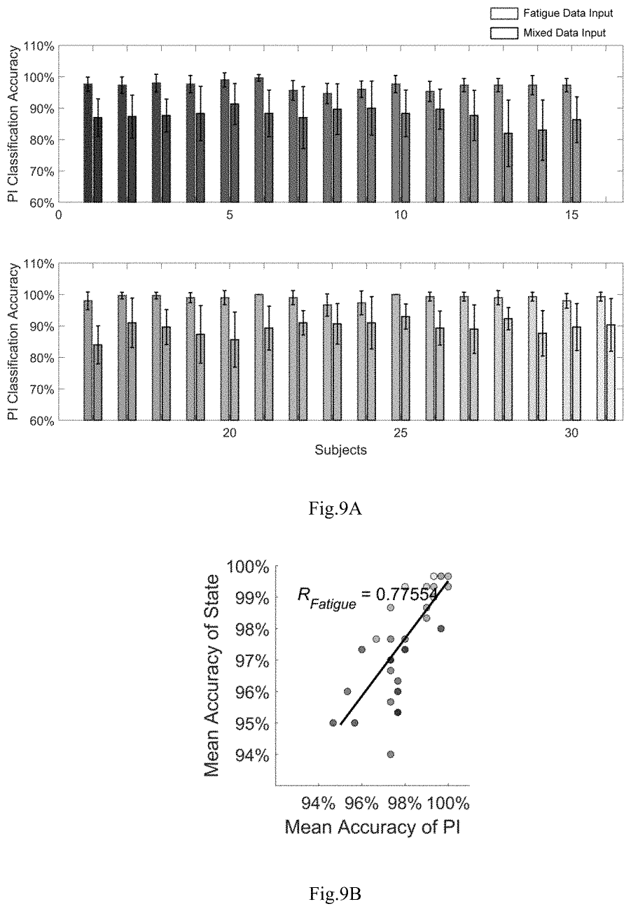

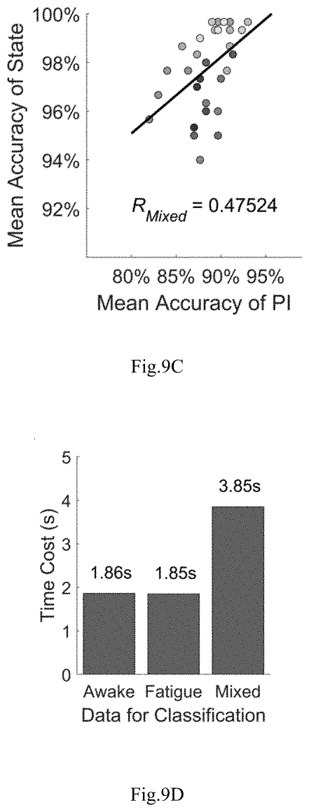

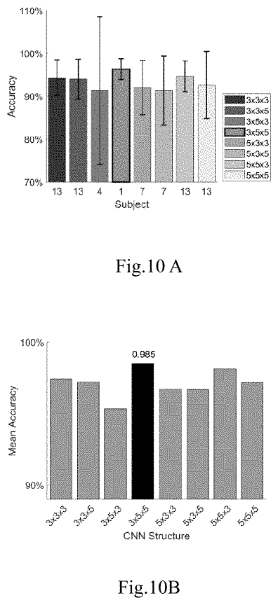

In some embodiments, the EEG signals are acquired and pre-processed in a manner similar to or identical to that used in the above training. Since it is the classification task of the actual application, the foregoing label is not added at this point (that is, the data is classified and marked for training purposes, so as to facilitate the testing and verification of test results). The classification effect is also shown in FIGS. 4-10.

In some embodiments, each EEG signal in the EEG data set is input into a first attention-mechanism-based convolutional neural network, and pattern features for identifying a driver ID PI are extracted from the EEG data; and/or each EEG signal in the EEG data set is input into a second attention-mechanism-based convolutional neural network, and pattern features for identifying an awake state and a fatigue state of a driver are extracted. The first attention-mechanism-based convolutional neural network can be the first EEG recognition model, while the second attention-mechanism-based convolutional neural network can be the second EEG recognition model. The two models may have the same network structure, for different classification tasks, some parameters in the model are different, and the two models may share input data, namely, EEG signals from multiple EEG signal sensors, to solve the multitask classification.

In some embodiments, the step of classifying the pattern features of the EEG data to obtain an EEG pattern classification result includes:

inputting feature vectors of the pattern features of the EEG data and outputting the EEG pattern classification result by using a Softmax classifier, wherein a function h_{θ}(x) of the Softmax classifier is constructed as:

$${h }_{θ} ( x \text{?} ) = [ \begin{matrix}p ( y \text{?} = 1 \vert x \text{?} ; θ ) \\ ⋮ \\ p ( y \text{?} = k \vert x \text{?} ; θ )\end{matrix} ] = \frac{1}{{\sum }_{j = 1}^{k} e \text{?}} [ \begin{matrix}e \text{?} \\ ⋮ \\ {e}^{\text{?}}\end{matrix} ]$$

$$\text{?} \text{indicates text missing or illegible when filed}$$

where θ₁, θ₂, . . . θₖϵ [symbol] ⁿ⁺¹ denote the model parameters,

$$\frac{1}{{\sum }_{j = 1}^{k} e \text{?}}$$

$$\text{?} \text{indicates text missing or illegible when filed}$$

normalized the probability distribution so that the summation of probability is 1. The one with a higher probability was used as the classification result of the test.

According to yet another embodiment of the present disclosure, a system for classifying EEG patterns is further proposed, including: a memory; a processor; a sensor connected to the processor, configured to detect the EEG signals; and a computer program stored in the memory and executed by the processor, wherein when the processor executes the computer program, the method for classifying EEG patterns is implemented according to the EEG signals detected by the sensor.

In some embodiments, the system for classifying EEG patterns or the EEG pattern classification model of the present disclosure can be stored as a logical sequence in a computer-readable storage medium, or can be written to a chip and the chip can be installed in a driving electronic device.

Different from the experimental environment, at this point, the driver is sitting in a real vehicle, a customized or commercially available helmet or a wearable device may be provided so as to make at least one sensor placed on the scalp, to collect the EEG signals, the helmet or wearable device may communicate with the processor in a wired or wireless manner, or communicate with the driving electronic device installed with the chip.

The EEG signals, as a means of studying the brain, have attracted more and more interest with the development of deep learning.EEG signals during driving is used to conduct PI. Therefore, the influence to EEG signal are relatively simple and confined in the person's fatigue state and the driving condition. And each subject will undoubtedly press the brake pedal while facing danger. Although the original intention of the experiment aims to classify driving fatigue state rather than PI. And finally, the same network structure is used for the classification of both driving fatigue state and PI. According to the experiment, it is found that the same network structure can be used to classify the driving fatigue state and PI.

Based on the collected data, the performance of the two methods are compared and it is found that the proposed method had a high classification accuracy and a short training time for both PI and driving fatigue state (Error! Reference source not found.4 and Error! Reference source not found.5). Third, both of the two experiments use a small number of electrodes for classification. Although the highest mean accuracy of PI cannot reach 99%, the lowest averaged mean accuracy of PI can be higher than 80%. Moreover, the accuracy of driving fatigue state can be as high as 94% with EEG data collection from frontal area. Fourth, our experiment is more practical. Nevertheless, although our experiment is based on driving simulator, the experiment in real driving condition provided that the safety and the convenience of using portable EEG data acquisition system could be improved.

According to some embodiments of the present disclosure, an ATT-CNN-based network is provided for driving-related multitask classification, which is related to the PI as well as the driving state with the same data. For PI and driving states, the average classification accuracy was as high as 98.5% and 98.2%, respectively. It can also make a good trade-off between classification accuracy and time cost. The results show that the network structure has potential application values in the multitask classification of biomedical signals.

It should be noted that the embodiments in this specification are all described in a progressive manner, for same or similar parts in the embodiments, reference may be made to these embodiments, and each embodiment focuses on a difference from other embodiments. Especially, device and system embodiments are basically similar to a method embodiment, and therefore are described briefly; for related parts, reference may be made to partial descriptions in the method embodiment. The described device and system embodiments are merely exemplary. The units described as separate parts may or may not be physically separate, and parts displayed as units may or may not be physical units, may be located in one position, or may be distributed on a plurality of network units. Some or all of the modules may be selected according to actual requirements to achieve the objectives of the solutions of the embodiments. A person of ordinary skill in the art may understand and implement the embodiments of the present invention without creative efforts.

It should be understood by those skilled in the art that functional modules or units in all or part of the steps of the method, the system and the apparatus disclosed above may be implemented as software, firmware, hardware and appropriate combinations thereof. In the hardware implementation, the division of functional modules or units mentioned in the above description may not correspond to the division of physical components. For example, one physical component may have multiple functions, or one function or step may be executed jointly by several physical components. Some or all components may be implemented as software executed by processors such as digital signal processors or microcontrollers, hardware, or integrated circuits such as application specific integrated circuits. Such software may be distributed on computer-readable media, which may include computer storage media (or non-transitory media) and communication media (or transitory media). As is known to those skilled in the art, the term, computer storage media, includes volatile and nonvolatile, removable and non-removable media implemented in any method or technology for storing information (such as computer-readable instructions, data structures, program modules or other data). The computer storage media include, but are not limited to, random access memory (RAM), read-only memory (ROM), electrically erasable programmable read-only memory (EEPROM), flash memory or other memory technologies, compact disc read-only memory (CD-ROM), digital versatile disc (DVD), or other optical disc storage, magnetic cassette, magnetic tape, magnetic disk storage or other magnetic storage devices, or any other media configured for storing desired information and accessible by the computer. In addition, as is known to those skilled in the art, communication media generally include computer-readable instructions, data structures, program modules or other data in modulated data signals such as carriers or other transmission mechanisms, and may include any information delivery medium.

Although the implementation modes disclosed by the present application are as described above, the content thereof is merely embodiments for facilitating the understanding of the present application and is not intended to limit the present application. Any person skilled in the art to which the present application pertains may make any modifications and changes in the forms and details of the implementation without departing from the spirit and scope disclosed by the present application, but the patent protection scope of the present application is still subject to the scope defined by the appended claims.

We claim:

1. A training method for an electroencephalogram (EEG) pattern classification model, comprising:
acquiring EEG data, pre-processing the EEG data, and labeling the EEG data to obtain a labeled training data set, wherein the training data set comprises the pre-processed and labeled EEG data;
inputting each piece of EEG data in the training data set into an attention-mechanism-based convolutional neural network to extract pattern features of the EEG data; and
modifying parameters for the EEG pattern classification model according to the pattern features and labels of the EEG data.

2. The training method for an EEG pattern classification model according to claim 1, wherein the attention-mechanism-based convolutional neural network comprises: at least one convolution layer; at least one max-pooling layer; an attention module; and a fully connected layer;
wherein the inputting each piece of EEG data in the training data set into an attention-mechanism-based convolutional neural network to extract pattern features of the EEG data comprises steps:
inputting each piece of EEG data into the at least one convolution layer, and extracting the pattern features of the EEG data to obtain a convolution feature vector comprising the pattern features;
inputting the convolution feature vector into the at least one max-pooling layer for pooling to obtain a pooled feature vector;
inputting the pooled feature vector into the attention module to calculate a normalized weight for the pooled feature vector and the summation of information reflecting the pattern features of the EEG data; and
outputting the pattern features of the EEG data through the fully connected layer.

3. The training method for an EEG pattern classification model according to claim 2, wherein the attention module performs the following calculations:
$$\begin{matrix} {u}_{i} = tanh ( W \text{?} h \text{?} + b \text{?} ) \\ {a}_{i} = \frac{exp ( {u}_{i}^{T} u \text{?} )}{{\sum }_{i} exp ( {u}_{i}^{T} u \text{?} )} \\ v = {\sum }_{i} {a}_{i} {h}_{i} \\ \text{?} \text{indicates text missing or illegible when filed}\end{matrix}$$
wherein bₛ is a bias; uᵢ is a hidden representation of hᵢ which is fed through a one-layer perceptron with a weight Wₛ; αᵢ is a normalized weight which is measured by the similarity of uᵢ with uₛ; uₛ is a hidden representation of another piece of EEG signal v is the summation of the all information of EEG signals.

4. The training method for an EEG pattern classification model according to claim 1, wherein the acquiring EEG data, pre-processing the EEG data, and labeling the EEG data to obtain a labeled training data set comprises steps:
acquiring EEG signals from multiple EEG signal sensors;
obtaining a multi-channel EEG signal by performing band-pass filtering and Fast ICA on the EEG signals;
digitizing and segmenting the multi-channel EEG signal according to a preset sampling rate and duration to obtain an EEG data set comprising multiple multi-channel EEG signal digital segments;
adding at least one label to each multi-channel EEG signal digital segment in the EEG data set to obtain labeled EEG data, wherein the label comprises an awake state, a fatigue state, and a driver ID; and
obtaining the labeled training data set.

5. The training method for an EEG pattern classification model according to claim 4, wherein
training a first EEG recognition model based on the labeled training data set with at least the driver ID label, wherein the first EEG recognition model is configured to identify and classify a driver ID PI based on EEG pattern features of a driver; and/or
training a second EEG recognition model based on the labeled training data set with at least the awake state and fatigue state labels, wherein the second EEG recognition model is configured to identify and classify awake-state and fatigue-state pattern features of the driver based on the EEG pattern features of the driver.

6. The training method for an EEG pattern classification model according to claim 5, wherein
the attention-mechanism-based convolutional neural network further comprises: a Softmax classifier set after the fully connected layer, configured to classify the driver ID PI; and/or classify the awake-state and fatigue-state pattern features of the driver, wherein feature vectors of the pattern features of the EEG data are input to the Softmax classifier, and EEG pattern classification results are output after calculation with a function h_{θ}(x) of the Softmax classifier, wherein the function h_{θ}(x) of the Softmax classifier is expressed as:
$${h }_{θ} ( x \text{?} ) = [ \begin{matrix}p ( y \text{?} = 1 \vert x \text{?} ; θ ) \\ ⋮ \\ p ( y \text{?} = k \vert x \text{?} ; θ )\end{matrix} ] = \frac{1}{{\sum }_{j = 1}^{k} e \text{?}} [ \begin{matrix}e \text{?} \\ ⋮ \\ {e}^{\text{?}}\end{matrix} ]$$
$$\text{?} \text{indicates text missing or illegible when filed}$$
wherein x is a function input, θ₁, θ₂ . . . θ_{R}ϵ [symbol] [symbol] +1 denotes parameters for extracting features, k is a classification dimension, and
$$\frac{1}{{\sum }_{j = 1}^{k} e \text{?}}$$
$$\text{?} \text{indicates text missing or illegible when filed}$$
is used to normalize probability distribution so as to ensure that the summation of probability values p equal to 1, wherein the value with higher probability is taken as a classification result; and
further comprises a cross-entropy loss function L, expressed as:
$$L = - \sum \text{?} y \text{?} log ( {h}_{θ} ( x \text{?} ) ) , \\ \text{?} \text{indicates text missing or illegible when filed}$$
wherein y is an output vector, and h_{θ} is the probability of belonging to a classification result.

7. A method for classifying electroencephalogram (EEG) patterns, comprising:
acquiring EEG signals, and pre-processing the EEG signals to obtain an EEG data set, wherein the EEG data set comprises the pre-processed EEG signals;
inputting each EEG signal in the EEG data set into an attention-mechanism-based convolutional neural network to extract pattern features of the EEG data; and
classifying the pattern features of the EEG data to obtain an EEG pattern classification result.

8. The method for classifying EEG patterns according to claim 7, wherein the inputting each EEG signal in the EEG data set into an attention-mechanism-based convolutional neural network to extract pattern features of the EEG data comprises:
inputting each EEG signal in the EEG data set into a first attention-mechanism-based convolutional neural network, and extracting from the EEG data to obtain pattern features for identifying a driver ID PI; and/or
inputting each EEG signal in the EEG data set into a second attention-mechanism-based convolutional neural network, and extracting from the EEG data to obtain pattern features for identifying an awake state and a fatigue state of a driver.

9. The method for classifying EEG patterns according to claim 8, wherein the classifying the pattern features of the EEG data to obtain an EEG pattern classification result comprises:
inputting feature vectors of the pattern features of the EEG data and outputting the EEG pattern classification result by using a Softmax classifier, wherein a function h_{θ(x) of the Softmax classifier is constructed as:}
$${h }_{θ} ( {x}^{i} ) = [ \begin{matrix}p ( {y}^{i} = 1 \vert {x}^{i} ; θ ) \\ ⋮ \\ p ( {y}^{i} = k \vert {x}^{i} ; θ )\end{matrix} ] = \frac{1}{{\sum }_{j = 1}^{k} {e}^{{θ}_{j}^{T} {x}^{i}}} [ \begin{matrix}{e}^{{θ}_{1}^{T} {x}^{i}} \\ ⋮ \\ {e}^{{θ}_{k}^{T} {x}^{i}}\end{matrix} ]$$
wherein x is a function input, θ₁, θ₂ . . . θ_{R}ϵ [symbol] ⁿ⁺¹ denotes parameters for extracting features, k is a classification dimension, and
$$\frac{1}{{\sum }_{j = 1}^{k} e \text{?}}$$
$$\text{?} \text{indicates text missing or illegible when filed}$$
is used to normalize probability distribution so as to ensuare that the summation of probability values p equal to 1, wherein the value with higher probability is taken as a classification result.

10. A system for classifying electroencephalogram (EEG) patterns, comprising:
a memory;
a processor;
a sensor connected to the processor, configured to detect the EEG signals according to claim 7; and
a computer program stored in the memory and runnable on the processor,
wherein when the processor executes the computer program, the method for classifying EEG patterns according to claim 7 is implemented according to the EEG signals detected by the sensor.
